(19) United States
(12) Patent Application Publication
(10) Pub. No.: US 2026/0327178 A1
(43) Pub. Date: Oct. 22, 2026

(54) PROCESS APPARATUS FOR AND METHOD OF HETEROAZEOTROPIC EXTRACTIVE DISTILLATION PROCESS

(71) Applicant: CHEMPOLIS OY, Oulu (FI)

(72) Inventor: Juhani KANTOMAA, Oulu (FI)

(21) Appl. No.: 19/486,330

(22) PCT Filed: May 21, 2024
(86) PCT No.: PCT/FI2024/050247

§ 371 Date: Nov. 20, 2025

(30) Foreign Application Priority Data
May 22, 2023 (FI) 20235566

(51) Int. Cl. C07C 51/46; B01D 3/36; B01D 3/40; B01D 3/42; C07D 307/48

(52) U.S. Cl. CPC C07C 51/46; B01D 3/36; B01D 3/40; B01D 3/4294; C07D 307/48

(57) ABSTRACT

A process apparatus for a distillation process comprises a distillation column that processes material of a mixture of water, at least acetic acid and formic acid and furfural, furfural being used as a former of azeotrope and an extractant in the mixture. A flowing material channel is located between a lower section of the column and an upper section of the column, the flowing material channel comprising a solid material remover. A feed arrangement is connected with the flowing material channel between the lower section of the column and the solid material remover. The flowing material channel receives a stream of the processed material from the lower section of the column, a stream of precipitant from the feed arrangement, precipitation effect of the precipitant being based on water, and directs the mixture of the streams of precipitant and processed material to the solid material remover, which removes solid material caused by the precipitant from the mixture. The flowing material channel feeds a stream of the mixture, from which the solid material has been at least partly removed, to the upper section of the column.

### FIELD

The invention relates to a process apparatus for and a method of a heteroazeotropic extractive distillation process.

### BACKGROUND

In a heteroazeotropic extractive distillation process that is used to separate water, at least one organic acid, and furfural, furfural being capable of acting both as a former of azeotrope and as an extractant.

Furfural polymerizes easily in acid process conditions of a column of the heteroazeotropic extractive distillation process. The polymerized furfural, in turn, contaminates the column and forms blockages on surfaces such as distillation packing(s). The contamination may disturb a mechanical operation of the process and lower a quality of an end product. For example, dynamics of the process and a heat transfer coefficient of the structures and materials may change. To clean the process system requires the process operation to be stopped for a substantially long time and/or undesirably often, which shortens the process operation and causes economical losses. Improvement relating to contamination is looked for.

### BRIEF DESCRIPTION

The present invention seeks to provide an improvement in the measurements.

The invention is defined by the independent claims. Embodiments are defined in the dependent claims.

If one or more of the embodiments is considered not to fall under the scope of the independent claims, such an embodiment is or such embodiments are still useful for understanding features of the invention.

### LIST OF DRAWINGS

Example embodiments of the present invention are described below, by way of example only, with reference to the accompanying drawings, in which

FIG. 1 illustrates an example of a process apparatus for a heteroazeotropic extractive distillation process;

FIG. 2 illustrates an example of data processing; and

FIG. 3 illustrates of an example of a flow chart of a method of a heteroazeotropic extractive distillation process.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment.

The articles “a” and “an” give a general sense of entities, structures, components, compositions, operations, functions, connections or the like in this document. Note also that singular terms may include pluralities.

Single features of different embodiments may also be combined to provide other embodiments. Furthermore, words “comprising” and “including” should be understood as not limiting the described embodiments to consist of only those features that have been mentioned and such embodiments may also contain features/structures that have not been specifically mentioned. All combinations of the embodiments are considered possible if their combination does not lead to structural or logical contradiction.

The term “about” means that quantities or any numeric values are not exact and typically need not be exact. The reason may be tolerance, resolution, measurement error, rounding off or the like, or a fact that the feature of the solution in this document only requires that the quantity or numeric value is approximately that large. A certain tolerance is always included in real life quantities and numeric values.

It should be noted that while Figures illustrate various embodiments, they are simplified diagrams that only show some structures and/or functional entities. The connections shown in the Figures may refer to logical or physical connections. It is apparent to a person skilled in the art that the described apparatus may also comprise other functions and structures than those described in Figures and text. It should be appreciated that details of some functions, structures, and the signaling used for measurement and/or controlling are irrelevant to the actual invention. Therefore, they need not be discussed in more detail here.

The expression “azeotrope” refers to a mixture of substances wherein vapour and liquid compositions are identical in a phase equilibrium. The azeotrope corresponds to an extreme point (minimum, maximum or saddle point) in a boiling temperature isobar or in a vapour pressure isotherm.

The expression “azeotropic distillation” refers to either distillation of azeotropic mixtures or distillation wherein an azeotrope forming component (“entrainer”) is added to a process.

The expression “extractive distillation” refers to distillation wherein a fully soluble, azeotrope forming component (“entrainer”) boiling at a relatively high temperature is added to a distillation column above the actual feed flow. Entrainer increases the relative volatility of the components and enhances separation of components, which is the main target of distillation.

Extraction, per se, refers to a process wherein a desired substance in a mixture dissolves in a solvent while the rest of the substances are insoluble in said solvent. In the mixture, the substances are completely mixed up with one another and the mixture, per se, may contain end products of chemical reactions.

The expression “heteroazeotrope” refers to an azeotrope having, in addition to the vapour phase, two liquid phases present.

The expression “heteroazeotropic distillation” refers to either distillation of heteroazeotropic mixtures or distillation wherein a heteroazeotrope forming component (“entrainer”) is added to a process.

The expression “heteroazeotropic extractive distillation” refers to a combination of heteroazeotropic distillation and extractive distillation. The component to be added, boiling at a relatively high temperature, changes relative volatilities of components to be separated and boiling at a lower temperature, and it forms a low boiling azeotrope with any one of the remaining components.

A column is a tubular, often upright construction wherein substances fed thereto become completely mixed up together. Such a column may be used for separating different fluids from each other based on the properties of the substances in various conditions. A typical field of application is chemical industry. The column, per se, is known to those skilled in the art.

Furfural is used as an entrainer in distillation. Furfural forms polymers when in contact with acids in a hot temperature. The polymers are partly in a dissolved and partly in insoluble form. The dissolved polymers precipitate when they come into contact with water inside the distillation column. Precipitated polymers cause fouling of the column internals. The polymers are difficult to properly be removed from the column. In prior art it is also challenging to prevent polymers entering the column. This document proposes that recycled flow with dissolved polymers is mixed with water or water rich stream outside the column and precipitates are removed before the recycled flow with furfural is fed back to the column. A target is to avoid, restrict or slow down precipitation and fouling inside the column.

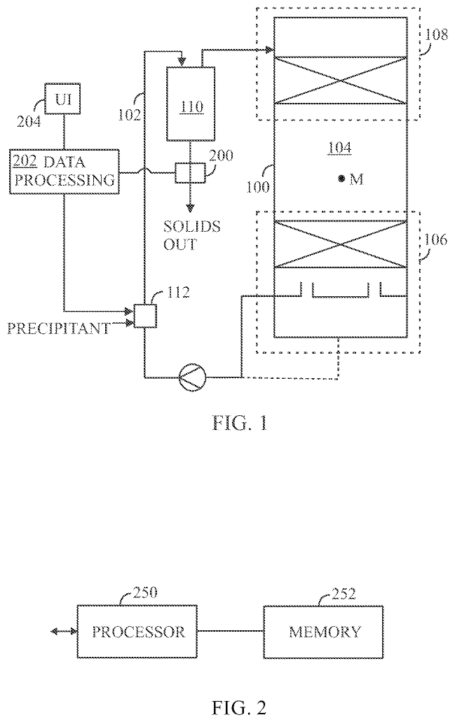

FIG. 1 shows an apparatus for a heteroazeotropic extractive distillation process. The heteroazeotropic extractive distillation process apparatus comprises at a distillation column 100 that processes material 104 of a mixture of water, at least acetic acid and formic acid and furfural. The furfural is used as a former of azeotrope and an extractant in the mixture within the distillation column 100. The process apparatus further comprises a flowing material channel 102 between a lower section 106 of the column 100 and an upper section 108 of the column 100. The flowing material channel 102 receives the processed material 104 from above a bottom sump of the distillation column 100 as a liquid stream. The flowing material channel 102 may be tube or a pipe, for example, within which the mixture from the distillation column 100 can flow. The upper section 108 refers to a part of the distillation column 100 that is longitudinally on one side of a middle point M of the column 100. When the distillation column 100 is in an erected position, the upper section 108 is above the middle point M. The lower section 106 refers to a part of the distillation column 100 that is longitudinally on an opposite side of a middle point M of the column 100 with respect to the upper section 108. When the distillation column 100 is in the erected position, the lower section 106 is below the middle point M. The lower section 106 may refer to a bottom of the column 104 as shown with dashed line.

The lower section 106 can be understood to be below a center of the column 100, where the center may be the same as the middle point M. The lower section 106 can be understood to reside below the distillation packing of the distillation column 100.

The flowing material channel 102 comprises a solid material remover 110. A feed arrangement 112 is connected with the flowing material channel 102 between the lower section of the column 100 and the solid material remover 110. That flowing material channel 102 comprises the solid material remover 110 means that the solid material remover 110 is connected between the distillation column 100 and the feed arrangement 112. Then the solid material remover 110 can remove fully or partially the solid material content of the flowing processed material within the flowing material channel 102 before it enters the distillation column 100.

The flowing material channel 102 receives a stream of the processed material 104 from the lower section 106 of the column 100. The flowing material channel 102 also receives a stream of precipitant from the feed arrangement 112. The precipitation effect of the precipitant is based on water. The flowing material channel 102 directs the mixture of the streams of precipitant and the processed material to the solid material remover 110. The solid material remover 110 removes solid material, which is formed under influence of the precipitant, from the mixture of the streams.

The flowing material channel 102 feeds a stream of the mixture, from which the solid material has been at least partly removed in the solid material remover 110, to the upper section 108 of the column 100.

In an embodiment, the solid material remover 110 comprises at least one of the following: a settling tank separator, a filter separator, a cyclone separator, a flotator, and an evaporation separator.

The settling tank separator may make the solid material to deposit on a bottom of the settling tank for feeding the stream of the mixture, from which the solid material has been at least partly removed, from an upper section of the settling tank, which is on an opposite side to the bottom, to the upper section 108 of the column 100.

The filter separator may comprise a filtering mesh that is configured to prevent solid material of the stream of the mixture 104 from propagating to upper section 108 of the column 100.

The cyclone separator may create a centrifugal force for separating the solid material from the stream of the mixture for feeding the stream of the mixture, from which the solid material has been at least partly removed to the upper section 108 of the column 100. The centrifugal force separates material of different density.

The evaporator may evaporate non-solid material of the mixture for removing the solid material from the stream of the mixture and feed the non-solid material to the upper section 108 of the column 100. A person skilled in the art is familiar with the solid material removers, per se.

In an embodiment, the distillation column 100 may cause and/or receive a pressure ranging between about 0.2 bar and about 2.5 bar inside the distillation column 100. When the distillation column 100 receives pressure, it means that the pressure within the distillation column 100 becomes that of the received pressure or is in a desirable manner related to the received pressure. The pressure may be measured with respect to a pressure outside the distillation column 100. In a corresponding manner, the distillation column 100 may cause or receive temperature ranging between about 40 °C and about 170 °C for an internal processing condition of the column 100. It means that the temperature within the distillation column 100 becomes that of the received temperature or is in a desired manner related to the received temperature. In an embodiment, the temperature within the distillation column 100 is between about 60 °C and about 170 °C, for example. Correspondingly, the temperature of the liquid stream fed from the solid material remover 110 into the distillation column 100 is between about 60 °C and about 140 °C, for example.

In an embodiment, the feed arrangement 112 may vary the feed of the precipitant to the flowing material channel 102 in response to the variation of solid material included in the stream of the material 104 of the distillation column 100 flowing in the material channel 102.

In an embodiment, the feed arrangement 112 may increase the feed of the precipitant in response to increased amount of solids material included in the stream of the material 104 of the distillation column 100 flowing in the material channel 102. In an embodiment, the feed arrangement 112 may be an adjustable valve, for example. In this embodiment, the feed arrangement 112 may be manually adjustable valve, for example. Then the feed arrangement 112 may receive a control action for adjusting a flow of the precipitant to the flowing material channel 102. The manually adjustable valve may operate electrically or manually. That is, the control action a user inputs to the feed arrangement 112 may be performed by turning a handwheel, a lever or other actuator that directly or indirectly adjusts the throttle of the feed arrangement 112. The direct adjustment may be mechanical, and the indirect adjustment may be at least pertly electrical, for example.

In an embodiment, the process apparatus may comprise a meter 200 that measures an amount of solids within the solid material remover 110 and/or discharged solids from the solid material remover 110. The meter 200 may be measure the solids optically. In an embodiment, the optical measurement may be based on scattering of light. In an embodiment, the optical measurement may be based on imaging and image processing. A person skilled in the art is familiar with measurement of solids in liquid, per se.

In an embodiment, the process apparatus may comprise a data processor 202 that receives data on the amount of solids. Then the data processor 202 may control the feed arrangement 112 to feed the precipitant to the flowing material channel 102 as a function of the received data on the solids. In this embodiment, the feed arrangement 112 may be electrically controllable valve, for example. A person skilled in the art is familiar with valves, per se.

FIG. 2 illustrates an example of the data processor 202 that comprises one or more processors 250 and one or more memories 252 including computer program code. The one or more memories 252 and the computer program code may, with the one or more processors 250, cause apparatus at least to receive data, output data and/or control the process apparatus.

The term “data processor” includes a computational device that performs logical and arithmetic operations. For example, a “data processor” may comprise an electronic computational device, such as an integrated circuit, a microprocessor, a mobile computing device, a laptop computer, a tablet computer, a personal computer, or a mainframe computer. A “data processor” may comprise a central processing unit, an ALU (arithmetic logic unit), a memory unit, and a control unit that controls actions of other components of the computer so that steps of a computer program are executed in a desired sequence. A “data processor” may also include at least one peripheral unit that may include an auxiliary memory (such as a disk drive or flash memory), and/or may include data processing circuitry.

A user interface 204 means an input/output device and/or unit. Non-limiting examples of a user interface include a touch screen, other electronic display screen, keyboard, mouse, microphone, handheld electronic controller, digital stylus, display screen, speaker, and/or projector for projecting a visual display.

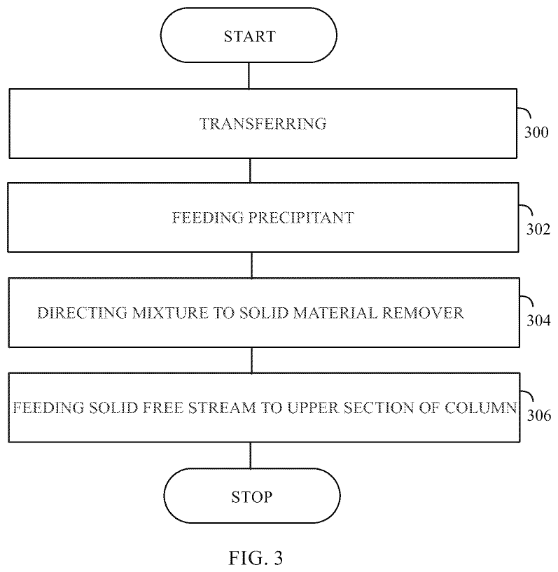

FIG. 3 illustrates an example of a method of a heteroazeotropic extractive distillation process. A distillation column 100 processes material 104 of a mixture of water, at least acetic acid and formic acid and furfural, furfural being a former of azeotrope and an extractant in the process of the distillation column 100.

In step 300, a stream of material 104 processed in the distillation column 100 is transferred through a flowing material channel 102 between a lower section 106 of the column 100 and an upper section 108 of the column 100, the flowing material channel 102 comprising a solid material remover 110.

In step 302, a stream of precipitant is fed to the flowing material channel 102 by a feed arrangement 112, which is connected with the flowing material channel 102 between the lower section of the column 100 and the solid material remover 110, a precipitation effect of the precipitant being based on water.

In step 304, a mixture of the streams of precipitant and processed material 104 is directed to the solid material remover 110, which removes solid material formed by the precipitant from the stream of the material 104.

In step 306, a stream of the material 104, from which the solid material has been at least partly removed, is fed to the upper section 108 of the column 100. The stream fed to the upper section 108 of the distillation column 100 includes furfural, for example.

It will be obvious to a person skilled in the art that, as technology advances, the inventive concept can be implemented in various ways. The invention and its embodiments are not limited to the example embodiments described above but may vary within the scope of the claims.

1-10. (canceled)

11. A process apparatus for a heteroazeotropic extractive distillation process, which comprises a distillation column that is configured to process material of a mixture of water, at least acetic acid and formic acid and furfural, furfural being used as a former of azeotrope and an extractant in the mixture, where the process apparatus comprises
process apparatus is configured to process said water, at least one organic acid, and furfural in pressure ranging between 0.2 bar and 2.5 bar and temperature ranging between 40 °C and 200 °C for an internal processing condition of the column, which form insoluble polymers of furfural causing fouling of the column internals; and the process apparatus further comprises
a flowing material channel between a lower section of the column and an upper section of the column, the flowing material channel comprising a solid material remover, and
a feed arrangement connected with the flowing material channel between the lower section of the column and the solid material remover;
the flowing material channel is configured to receive a stream of the processed material with dissolved polymers of furfural from the lower section of the column, receive a stream of precipitant from the feed arrangement for mixing water or water rich stream and the dissolved polymers outside the column, precipitation effect of the precipitant to the dissolved polymers of furfural being based on water, and direct the mixture of the streams of precipitant and processed material to the solid material remover, which is configured to remove solid material of insoluble dissolved polymers of furfural formed by the precipitant from the stream of the material before it is fed back to the column; and
the flowing material channel is configured to feed a stream of the mixture, from which the solid material has been at least partly removed, to the upper section of the column.

12. The apparatus of claim 11, wherein the solid material remover comprises at least one of the following: a settling tank separator; a filter separator, a cyclone separator and an evaporation separator, and
the settling tank separator being configured the make the solid material to descent to a bottom of the settling tank for feeding the stream of the mixture, from which the solid material has been at least partly removed, from an upper section of the settling tank, which is on an opposite side to the bottom, to the upper section of the column;
the filter separator comprises a filtering mesh that is configured to prevent solid material of the stream of the mixture from propagating to upper section of the column;
the cyclone separator is configured to create a centrifugal force for separating the solid material from the stream of the mixture for feeding the stream of the mixture, from which the solid material has been at least partly removed to the upper section of the column; and
the evaporator being configured to evaporate non-solid material of the mixture for removing the solid material from the stream of the mixture and feed the non-solid material to the upper section of the column.

13. The apparatus of claim 11, wherein the feed arrangement is configured to vary the feed of the precipitant to the flowing material channel in response to the variation of solid material included in the material flowing in the material channel.

14. The apparatus of claim 13, wherein the feed arrangement is configured to increase the feed of the precipitant in response to an increased amount of solids material included in the material flowing in the material channel.

15. The apparatus of claim 11, wherein apparatus comprises a meter that is configured to measure an amount of solids within the solid material remover and/or discharged solids from the solid material remover.

16. The apparatus of claim 15, wherein the apparatus comprises a data processor that is configured to receive data on the amount of solids, and control the feed arrangement to feed the precipitant to the flowing material channel as a function of the received data on the solids.

17. A method of a heteroazeotropic extractive distillation process, wherein comprises a distillation column that processes material of a mixture of water, at least acetic acid and formic acid and furfural, furfural being a former of azeotrope and an extractant in the mixture, the method comprising
processing said water, at least one organic acid, and furfural in pressure ranging between 0.2 bar and 2.5 bar and temperature ranging between 40 °C and 200 °C for an internal processing condition of the column, which forms insoluble polymers of furfural and cause fouling of the column internals; and
transferring a stream of the material processed in the distillation column through a flowing material channel between a lower section of the column and an upper section of the column, the flowing material channel comprising a solid material remover;
feeding a stream of precipitant to the flowing material channel including dissolved polymers of furfural by a feed arrangement, which is connected with the flowing material channel between the lower section of the column and the solid material remover for mixing water or water rich stream and the dissolved polymers outside the column, a precipitation effect of the precipitant to the dissolved polymers of furfural being based on water;
directing a mixture of the streams of precipitant and processed material to the solid material remover, which removes solid material of insoluble polymers of furfural formed by the precipitant from the stream of the material before it is fed back to the column; and
feeding a stream of the material, from which the solid material has been at least partly removed, to the upper section of the column.

18. The method of claim 17, the method further comprising the solid material remover comprises at least one of the following: a settling tank separator; a filter separator, a cyclone separator and an evaporation separator, and
making, within the settling tank separator, the solid material to descent to a bottom of the settling tank for feeding the stream of the mixture, from which the solid material has been at least partly removed, from an upper section of the settling tank, which is on an opposite side to the bottom, to the upper section of the column;
preventing, by the filter separator that comprises a mesh, solid material of the stream of the mixture from propagating to upper section of the column;
separating the solid material from the stream of the mixture by the cyclone separator that creates a centrifugal force for the separation for feeding the stream of the mixture, from which the solid material has been at least partly removed to the upper section of the column; and
evaporating, by the evaporator, non-solid material of the mixture for removing the solid material from the stream of the mixture and feed the non-solid material to the upper section of the column.
